(19) United States
(12) Patent Application Publication
(10) Pub. No.: US 2002/0115627 A1
(43) Pub. Date: Aug. 22, 2002

(54) Phosphatonin-related gene and methods of use thereof

(76) Inventors: Susan Schiavi, Hopkinton, MA (US); Stephen L. Madden, Sudbury, MA (US); Parthasarathy Manavalan, Medway, MA (US); Michael Levine, Owings Mills, MD (US); Suzanne Jan de Beur, Baltimore, MD (US)

Correspondence Address: Antoinette F. Konski, McCutchen, Doyle, Brown & Enersen, LLP, 18th Floor, Three Embarcadero Center, San Francisco, CA, 94111, US

(21) Appl. No.: 09/909,775

(22) Filed: Jul. 19, 2001

Related U.S. Application Data
(60) Provisional application No. 60/219,365, filed on Jul. 19, 2000.
(60) Provisional application No. 60/261,438, filed on Jan. 12, 2001.

(51) Int. Cl. A61K 48/00; C12Q 1/00; C12Q 1/68

(52) U.S. Cl. 514/44; 435/6; 435/4

(57) ABSTRACT

This invention provides methods for modulating phosphate homeostasis and renal phosphate transport by delivering agents that alter the expression of the FRP-4 gene or alter the activity of the FRP-4 protein. The methods of the invention are useful for modulating bone mineralization, renal phosphate transport, alleviating oncogenic osteomalacia-associated symptoms and treating phosphate homeostasis-related disease. The invention further provides methods for reducing phosphate re-absorption by delivering to a subject FRP-4 protein or polynucleotides that encode this protein. In addition, the invention provides methods for detecting and monitoring expression of the FRP-4 gene and modulating the phenotype of a neoplastic cell associated with oncogenic osteomalacia. Finally, the invention provides methods for screening candidate agents to identify compositions that modify the activity of the FRP-4 gene and protein.

### RELATED APPLICATIONS

This application claims the benefit under 35 U.S.C. 119(e) of provisional application serial No. 60/219,365 filed Jul. 19, 2000 and provisional application serial No. 60/261,438 filed Jan. 12, 2001, the disclosures of which are hereby incorporated by reference in their entirety.

### FIELD OF INVENTION

This invention relates to methods of using a gene encoding the frizzled related protein 4 (FRP-4) and the FRP-4polypeptide to treat phosphate transport related disease.

### BACKGROUND OF INVENTION

It is well known that many, but not all genes present in a cell are expressed at any given time. Fundamental questions of biology require knowledge of which genes are transcribed and the relative abundance of transcripts in different cells. Typically, when and to what degree a given gene is expressed has been analyzed one gene at a time.

Thus, information regarding the identity of all expressed genes in a cell and the level of expression of these genes would facilitate the study of many cellular processes such as activation, differentiation, aging, viral transformation, morphogenesis, and mitosis. A comparison of the expressed genes of a particular cell or the same cell from various individuals or species, under the same or different environmental stimuli, provides valuable insight into the molecular biology of the cell.

Phosphate plays a critical role in many cellular processes essential to normal functionality of a human body. Phosphate homeostasis is primarily regulated by kidney, largely through variation in renal tubular re-absorption of phosphate. Alterations of the phosphate transporting function of kidney and subsequent disturbance of serum phosphate concentration often lead to serious biochemical and clinical problems. Some diseases that are known to be associated with abnormal serum phosphate levels include inherent rickets in children, acquired osteomalacia in adults, rhabdomyolysis, cardiomyopathy, tumoral calcinosis or other renal failures and related secondary syndromes. Kumar (1997) Nephrol. Dial. Transplant. 12:11-13.

Of several known bone diseases associated with hypophosphatemia, X-linked hypophosphatemia rickets (HYP) and oncogenic osteomalacia (OOM) have been extensively studied in recent years. Rowe (1994) Hum. Genet. 94:457-467. HYP and OOM, although one inherited and the other acquired, have very similar clinical features. Both are characterized by symptoms such as hypophosphatemia, phosphaturia, and low serum concentrations of 1,25-dihydroxyvitamin D. Inadequate phosphate level leads to defective skeletal mineralization, which in turn causes deformed bones (rickets) or bone softening (osteomalacia).

Biological and clinical studies of hypophosphatemia- or hyperphosphatemia-related syndromes have been focused on understanding the molecular mechanism of phosphate uptake. Recent developments have provided several lines of evidence suggesting the existence of a humoral factor or factors specifically involved in renal phosphate transport regulation. Kumar (1997), supra. In HYP studies, a novel gene, PEX, has been associated with the hypophosphatemia phenotype. The PEX gene shares strong homology to a family of membrane-bound endopeptidases. Thus it is postulated that the PEX protein functions in cellular processing of putative hormone substrates. Rowe (1997) Exp. Nephrol. 5:355-363. OOM can be caused by a variety of histologically distinct tumors, mainly of mesenchymal origin (hemangiopericytomas). Removal of the tumor from the OOM patients promptly reverses defective symptoms and results in complete cure of the bone disease, indicating a tumor-secreted circulating factor capable of inhibiting renal phosphate transport.

Despite the well-documented circumstantial evidences for one or more humoral factors specifically involved in phosphate homeostasis regulation, the putative factor, “phosphatonin”, is yet to be identified. Several attempts to purify the factor or clone the gene encoding a protein having phosphatonin activity have been unsuccessful, in part due to difficulties in maintaining secretion levels of the putative factor in established tumor cultures.

In addition to phosphate regulation, it is likely that some effects of OOM as well as certain defects in bone mineralization are mediated by factors that directly interfere or promote bone metabolism as well as factors that mediate bone mineral homeostasis. The highly interactive pathways that govern phosphate metabolism and bone mineralization may also be influenced indirectly by polypeptide factors functioning to control protein synthesis, processing and secretion. For example, OOM induced changes may alter phosphate metabolism and bone mineralization by altering the balance of factors containing heparin sulfate or other glycosaminoglycans by increasing or decreasing the levels of lysosomal proteases. Such factors provide targets for improved therapies for a wide range of conditions including, osteoporosis, osteomalacia, rickets, hypophosphatasia, Falconin syndrome and renal osteodystrophy. Identification of such factors provides useful insights into phosphate regulation disorders.

There exits a need to identify genes differentially expressed in neoplastic cells associated with OOM, particularly those responsible for regulating renal phosphate transport. The analysis of gene expression pattern specific to the cells of interest not only leads to the identification of genes corresponding to phosphatonin activity, but also provide molecular information about gene activities related to other tumor-associated disease states. For example, tumor cells associated with OOM might be considered as a source of novel angiogenic factors or could be used to compare gene expression with different types of tumors. The identification of tumor-derived regulating factors can also help diagnosing and treating non-cancerous diseases with irregular phosphate homeostasis, such as renal failures and inherited rickets. Furthermore, the identification of factors that directly control bone formation and metabolism will provide important tools for therapeutic intervention in bone disease. Ultimately this will lead to the understanding of the mechanisms involved in phosphate metabolism and osteogenesis.

### SUMMARY OF THE INVENTION

This invention provides methods for modulating phosphate homeostasis and/or renal phosphate transport by delivering agents that alter the expression of the FRP-4 gene or alter the activity of the FRP-4 protein. The methods of the invention are useful for one or more of modulating bone mineralization, modulating renal phosphate transport, alleviating oncogenic osteomalacia-associated symptoms and treating phosphate homeostasis-related disease.

The invention further provides methods for reducing phosphate re-absorption by delivering to a subject FRP-4 protein or polynucleotides that encode the FRP-4 protein. In addition, the invention provides methods for detecting and monitoring expression of the FRP-4 gene.

In one embodiment, the invention provides methods for modulating the phenotype of a neoplastic cell associated with oncogenic osteomalacia by delivering an agent that alters the expression of the FRP-4 gene. Additionally, the invention provides methods for screening candidate agents to identify compositions that modify the activity of the FRP-4 gene and protein.

This invention provides isolated polynucleotides useful in the methods identified herein, such as polynucleotides encoding oncogenic osteomalacia-related genes (OOM) (e.g., FRP-4). Polynucleotides of the invention are intended to include DNA, cDNA, RNA and genomic DNA. Expression systems, including gene delivery vehicles such as liposomes and vectors, and host cells containing the polynucleotides are further provided by this invention.

The present invention also provides proteins encoded by the polynucleotides. In one embodiment, the proteins have oncogenic osteomalacia-related activity, which can be detected by using the methods described herein.

Additionally, nucleic acid probes and primers that hybridize to invention polynucleotides are provided, as well as isolated nucleic acids comprising unique, expressed gene sequences.

The present invention further includes antisense oligonucleotides, antibodies, hybridoma cell lines and compositions containing same.

The present invention also provides methods of monitoring gene expression using invention polynucleotides.

The methods of monitoring gene expression are useful for detecting a cell expressing oncogenic osteomalacia-related polypeptide and for detecting a neoplastic cell associated with oncogenic osteomalacia.

This invention further provides methods for modulating the expression of the inventive polynucleotides, for altering the activity of the proteins encoded by the polynucleotides, and for treating symptoms of phosphate transport related diseases and diseases characterized by abnormal bone mineralization. These diseases include but are not limited to, oncogenic osteomalacia, X-linked hypophosphataemia rickets, rhabdomyolysis, osteoporosis, cardiamyopathy, tumoral calcinosis, renal failure and bone mineralization.

This invention also provides a method for screening for candidate agents that modulate the expression of a polynucleotide of the invention or its complement, by contacting a test agent with a neoplastic cell associated with oncogenic osteomalacia and monitoring expression of the polynucleotide, wherein the test agent which modifies the expression of the polynucleotide is a candidate agent.

The present invention also provides assays for the isolation of the ligand or ligands capable of modulating the activity of the FRP-4 protein and therapeutic uses for said ligand.

This invention further provides assays for the assessment and development of candidate agents capable of modulating the activity of the FRP-4 protein and therapeutic uses for said candidate agent.

### BRIEF DESCRIPTION OF THE FIGURES

FIG. 1 shows SEQ ID NO. 1, a polynucleotide encoding the human frizzled-related protein, FRP-4. The polynucleotide contains 2840 nucleotides and has a reading frame that stretches from position 258 through 1298. There is an open reading frame from nucleotide 258 through 1295. The initiating ATG and termination codon are identified by bold type.

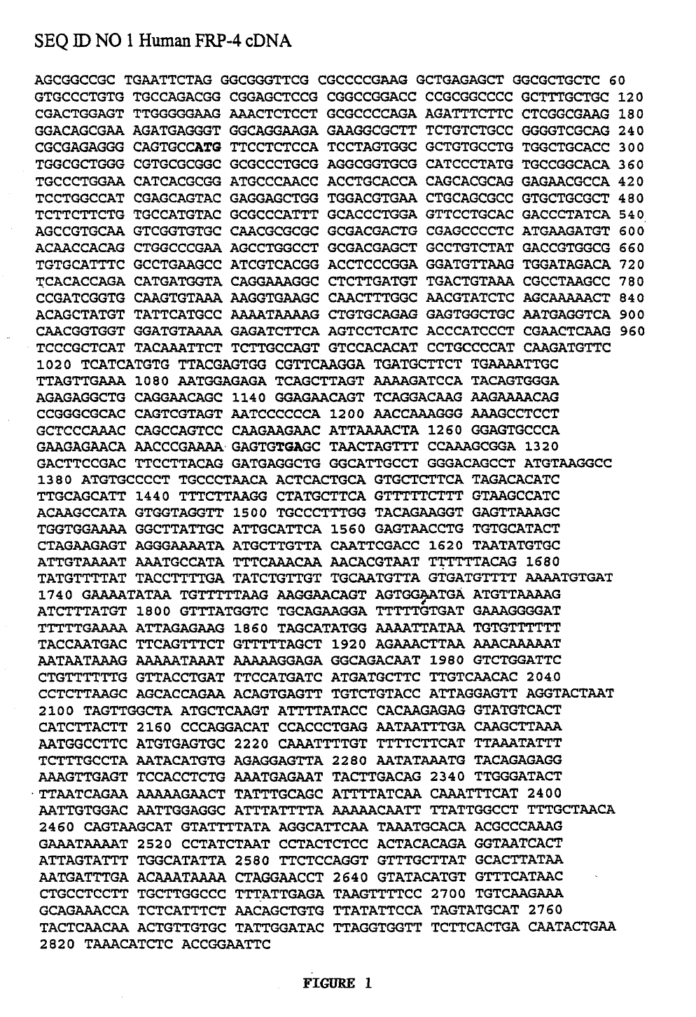

FIG. 2 shows the corresponding amino acid sequence (SEQ ID NO: 2).

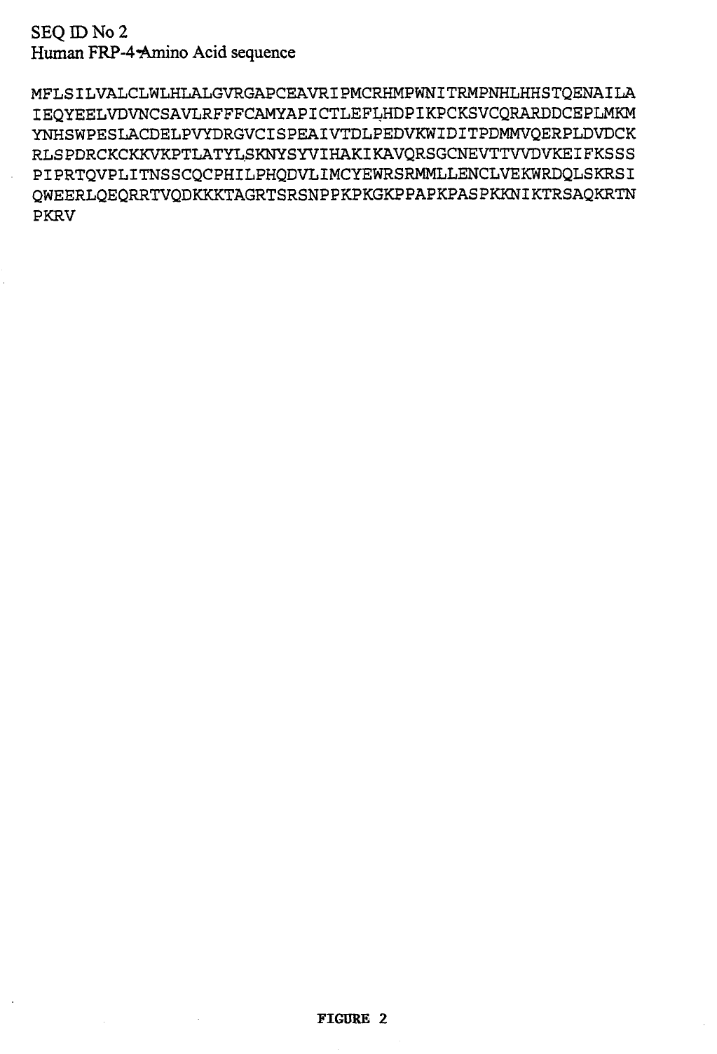

### DETAILED DESCRIPTION

Throughout this disclosure, various publications, patents and published patent specifications are referenced by an identifying citation. The disclosures of these publications, patents and published patent specifications are hereby incorporated by reference into the present disclosure to more fully describe the state of the art to which this invention pertains.

Definitions

The practice of the present invention will employ, unless otherwise indicated, conventional techniques of immunology, molecular biology, microbiology, cell biology and recombinant DNA. These methods are described in the following publications. See, e.g., Sambrook, et al. MOLECULAR CLONING: A LABORATORY MANUAL, 2^{nd} edition (1989); CURRENT PROTOCOLS IN MOLECULAR BIOLOGY (F. M. Ausubel, et al. eds., (1987)); the series METHODS IN ENZYMOLOGY (Academic Press, Inc.); “PCR: A PRACTICAL APPROACH” (M. MacPherson, et al., IRL Press at Oxford University Press (1991)); PCR 2: A PRACTICAL APPROACH (M. J. MacPherson, B. D. Hames and G. R. Taylor eds. (1995)); ANTIBODIES, A LABORATORY MANUAL (Harlow and Lane, eds. (1988)); and ANIMAL CELL CULTURE (R. I. Freshney, ed. (1987)).

As used in the specification and claims, the singular form “a”, “an” and “the” include plural references unless the context clearly dictates otherwise. For example, the term “a cell” includes a plurality of cells, including mixtures thereof.

The term “comprising” is intended to mean that the compositions and methods include the recited elements, but not excluding others. “Consisting essentially of” when used to define compositions and methods, shall mean excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions of this invention. Embodiments defined by each of these transition terms are within the scope of this invention.

The terms “polynucleotide” and “nucleic acid molecule” are used interchangeably to refer to polymeric forms of nucleotides of any length. The polynucleotides may contain deoxyribonucleotides, ribonucleotides, and/or their analogs. Nucleotides may have any three-dimensional structure, and may perform any function, known or unknown. The term “polynucleotide” includes, for example, single-, double-stranded and triple helical molecules, a gene or gene fragment, exons, introns, mRNA, tRNA, rRNA, ribozymes, cDNA, recombinant polynucleotides, branched polynucleotides, plasmids, vectors, isolated DNA of any sequence, isolated RNA of any sequence, nucleic acid probes, and primers. A nucleic acid molecule may also comprise modified nucleic acid molecules.

A “gene” refers to a polynucleotide containing at least one open reading frame that is capable of encoding a particular polypeptide or protein after being transcribed and translated.

An “FRP-4 gene” is a polynucleotide comprising an ordered sequence of nucleotides located in a particular position on a particular chromosome that encodes a frizzled-related protein-4 polypeptide, such as the amino acid sequence shown in SEQ ID NO 2. The term gene is intended to include contiguous polynucleotide sequences such as promoters and enhancers that modulate expression. As used herein the term FRP-4 gene refers to all orthologous sequences from divergent species, ie. homologous sequences encoding polypeptides that have the same activity in different species. It is particularly intended to include the FRP-4 genes of humans, simians and rodents.

An “FRP-4 polynucleotide” means any ordered sequence of polynucleotides that encode a frizzled-related protein-4 polypeptide, a portion of such a peptide, or a portion of the FRP-4 gene. An “FRP-4 polynucleotide” thus include cDNA's, probes, primers, and other molecules comprising polynucleotide sequences derived from the complete FRP-4 gene, eg. biologically equivalent polynucleotides.

Biologically equivalent polynucleotides are polynucleotides which differ from the polynucleotides described above, but produce the same phenotypic effect, such as the allele, splice variant and homolog. These altered, but phenotypically equivalent polynucleotides are referred to as “biologically equivalent polynucleotide” and “equivalent nucleic acids.” This methods of the invention also encompasses polynucleotides characterized by changes in non-coding regions that do not alter the phenotype of the polypeptide produced therefrom when compared to the polynucleotide herein. This invention further envisions the use of polynucleotides, which hybridize to the polynucleotides of the subject invention under conditions of moderate or high stringency.

Biologically equivalent polynucleotides useful in the methods of this invention are identified using sequence homology searches. Several embodiments of biologically equivalent polynucleotides are within the scope of this invention, e.g., those characterized by possessing at least 75%, or at least 80%, or at least 90% or at least 95% sequence homology as determined using a sequence alignment program under default parameters correcting for ambiguities in the sequence data, changes in nucleotide sequence that do not alter the amino acid sequence because of degeneracy of the genetic code, conservative amino acid substitutions and corresponding changes in nucleotide sequence, and variations in the lengths of the aligned sequences due to splicing variants or small deletions or insertions between sequences that do not affect function.

A variety of software programs are available in the art. Non-limiting examples of these programs are BLAST family programs including BLASTN, BLASTP, BLASTX, TBLASTN, and TBLASTX (BLAST is available from the worldwide web at http://www.ncbi.nlm.nih.gov/BLAST/), FastA, Compare, DotPlot, BestFit, GAP, FrameAlign, ClustalW, and PileUp. These programs can be obtained commercially in a comprehensive package of sequence analysis software such as GCG Inc.'s Wisconsin Package. Other similar analysis and alignment programs can be purchased from various providers such as DNA Star's MegAlign, or the alignment programs in GeneJockey. Alternatively, sequence analysis and alignment programs can be accessed through the world wide web at sites such as the CMS Molecular Biology Resource at http://www.sdsc.edu/ResTools/cmshp.html. Any sequence database that contains DNA or protein sequences corresponding to a gene or a segment thereof can be used for sequence analysis. Commonly employed databases include but are not limited to GenBank, EMBL, DDBJ, PDB, SWISS-PROT, EST, STS, GSS, and HTGS. Sequence similarity can be discerned by aligning the tag sequence against a DNA sequence database. Alternatively, the tag sequence can be translated into six reading frames; the predicted peptide sequences of all possible reading frames are then compared to individual sequences stored in a protein database such as s done using the BLASTX program.

Parameters for determining the extent of homology set forth by one or more of the aforementioned alignment programs are well established in the art. They include but are not limited to p value, percent sequence identity and the percent sequence similarity. P value is the probability that the alignment is produced by chance. For a single alignment, the p value can be calculated according to Karlin et al. (1990) PNAS 87: 2246. For multiple alignments, the p value can be calculated using a heuristic approach such as the one programmed in BLAST. Percent sequence identify is defined by the ratio of the number of nucleotide or amino acid matches between the query sequence and the known sequence when the two are optimally aligned. The percent sequence similarity is calculated in the same way as percent identity except one scores amino acids that are different but similar as positive when calculating the percent similarity. Thus, conservative changes that occur frequently without altering function, such as a change from one basic amino acid to another or a change from one hydrophobic amino acid to another are scored as if they were identical. A tag sequence is considered to lack substantial homology with any known sequences when the regions of alignment of comparable length exhibit less than 30% of sequence identity, more preferably less than 20% identity, even more preferably less than 10% identity.

Based on the known sequence of the FRP-4 gene, fragments of the gene or the full length coding sequence of the corresponding transcript or gene can be identified using various cloning methods known to artisans in the art. Polynucleotides useful for practicing the methods of the invention can comprise additional sequences, such as additional coding sequences within the same transcription unit, controlling elements such as promoters, ribosome binding sites, and polyadenylation sites, additional transcription units under control of the same or a different promoter, sequences that permit cloning, expression, and transformation of a host cell, and any such construct as may be desirable to provide embodiments of this invention.

A “FRP-4 polypeptide” is a molecule comprising an ordered sequence of amino acids specified by translation of a FRP-4 cDNA, such as is shown in SEQ ID NO 2. The term is used to refer to the complete FRP-4 amino acid sequence of FRP-4 (SEQ ID NO 2) as well as to alternatively spliced polypeptide molecules, and other portions of the complete molecule, such as protease cleavage products and synthetic peptides derived from the complete sequence. It also refers to orthologous FRP-4 polypeptides derived from various species including, but not limited to humans, simians, and rodents.

A “gene product” refers to the amino acid (e.g., peptide or polypeptide) generated when a gene is transcribed and translated.

A “sequence tag” or “tag” or “SAGE tag” is a short oligonucleotide containing defined nucleotide sequence that occurs in a certain position of a gene transcript. The length of a tag is generally under about 20 nucleotides, preferably between 9 to 15 nucleotides, and more preferably 10 nucleotides. The tag can be used to identify the corresponding transcript and gene from which it was transcribed. A tag can further comprise exogenous nucleotide sequences to facilitate the identification and utility of the tag. Such auxiliary sequences include, but are not limited to, restriction endonuclease cleavage sites and well known primer sequences for sequencing and cloning.

A sequence is the complement or is complementary to another sequence if they are related by the base-pairing rules. For example, in DNA, a sequence A-G-T in one strand is complementary to T-C-A in the other strand. A given sequence defines the complementary sequence.

As used herein, the term “modulate” means to alter or modify a process or biological function associated with, for example, phosphate homeostasis, renal phosphate transport, bone mineralization, and oncogenic osteomalacia-or its associated symptoms.

The term “peptide” is used in its broadest sense to refer to a compound of two or more subunit amino acids, amino acid analogs, or peptidomimetics. The subunits may be linked by peptide bonds. In another embodiment, the subunit may be linked by other bonds, e.g. ester, ether, etc. As used herein the term “amino acid” refers to either natural and/or unnatural or synthetic amino acids, including glycine and both the D or L optical isomers, and amino acid analogs and peptidomimetics. A peptide of three or more amino acids is commonly called an oligopeptide if the peptide chain is short. If the peptide chain is long, the peptide is commonly called a polypeptide or a protein.

The term “cDNAs” refers to complementary DNA, that is mRNA molecules present in a cell or organism made in to cDNA with an enzyme such as reverse transcriptase. A “cDNA library” is a collection of all of the mRNA molecules present in a cell or organism, all turned into cDNA molecules with the enzyme reverse transcriptase, then inserted into “vectors”.

A “probe” when used in the context of polynucleotide manipulation refers to an oligonucleotide that is provided as a reagent to detect a target potentially present in a sample of interest by hybridizing with the target. Usually, a probe will comprise a label or a means by which a label can be attached, either before or subsequent to the hybridization reaction. Suitable labels include, but are not limited to radioisotopes, fluorochromes, chemiluminescent compounds, dyes, and proteins, including enzymes.

A “primer” is a short polynucleotide, generally with a free 3′ —OH group that binds to a target or “template” potentially present in a sample of interest by hybridizing with the target, and thereafter promoting polymerization of a polynucleotide complementary to the target. A “polymerase chain reaction” (“PCR”) is a reaction in which replicate copies are made of a target polynucleotide using a “pair of primers” or a “set of primers” consisting of an “upstream” and a “downstream” primer, and a catalyst of polymerization, such as a DNA polymerase, and typically a thermally-stable polymerase enzyme. Methods for PCR are well known in the art, and taught, for example in “PCR: A PRACTICAL APPROACH” (M. MacPherson et al., IRL Press at Oxford University Press (1991)). All processes of producing replicate copies of a polynucleotide, such as PCR or gene cloning, are collectively referred to herein as “replication.” A primer can also be used as a probe in hybridization reactions, such as Southern or Northern blot analyses. Sambrook et al., supra.

A “promoter” is a region on a DNA molecule to which an RNA polymerase binds and initiates transcription. In an operon, the promoter is usually located at the operator end, adjacent but external to the operator. The nucleotide sequence of the promoter determines both the nature of the enzyme that attaches to it and the rate of RNA synthesis.

The term “genetically modified” means containing and/or expressing a foreign gene or nucleic acid sequence which in turn, modifies the genotype or phenotype of the cell or its progeny. “Foreign nucleic acid” includes, but is not limited to promoters, enhancers and gene activators. For example, a genetically modified cell includes a cell that contains a polynucleotide encoding FRP-4 polypeptide in its native environment but not expressed and expression has been turned on or the level of expression has been enhanced or lowered by the upstream insertion of a gene activator.

As used herein, “expression” or “expressed” refers to the process by which polynucleotides are transcribed into mRNA or by which transcription is enhanced. In another embodiment, the RNA is translated into peptides, polypeptides, or proteins. If the polynucleotide is derived from genomic DNA, expression may include splicing of the mRNA, if an appropriate eukaryotic host is selected.

“Hybridization” refers to a reaction in which one or more polynucleotides react to form a complex that is stabilized via hydrogen bonding between the bases of the nucleotide residues. The hydrogen bonding may occur by Watson-Crick base pairing, Hoogstein binding, or in any other sequence-specific manner. The complex may comprise two strands forming a duplex structure, three or more strands forming a multi-stranded complex, a single self-hybridizing strand, or any combination of these. A hybridization reaction may constitute a step in a more extensive process, such as the initiation of a PCR reaction, or the enzymatic cleavage of a polynucleotide by a ribozyme.

Hybridization reactions can be performed using traditional hybridization techniques under different stringency. In general, a low stringency hybridization reaction is carried out at about 40° C. in 10× SSC or a solution of equivalent ionic strength/temperature. A moderate stringency hybridization is typically performed at about 50° C. in 6× SSC, and a high stringency hybridization reaction is generally performed at about 60° C. in 1× SSC. Alternatively, TMAC hybridization technology can be used for hybridization reactions probed with pooled oligonucleotides such as the SAGE tags. The advantage of using TMAC hybridization is that the reaction condition is not dependent on the G+C content of the oligonucleotide, and the melting temperature is determined only by the length of the oligomers to be used.

When hybridization occurs in an anti-parallel configuration between two single-stranded polynucleotides, the reaction is called “annealing” and those polynucleotides are described as “complementary”. A double-stranded polynucleotide can be “complementary” or “homologous” to another polynucleotide, if hybridization can occur between one of the strands of the first polynucleotide and the second. “Complementarity” or “homology” (the degree that one polynucleotide is complementary with another) is quantifiable in terms of the proportion of bases in opposing strands that are expected to form hydrogen bonding with each other, according to generally accepted base-pairing rules. A polynucleotide that is 100% complementary to a second polynucleotide are understood to be “complements” of each other.

A “composition” is intended to mean a combination of active agent and another compound or composition, inert (for example, a detectable agent or label or a pharmaceutically acceptable carrier) or active, such as an adjuvant.

A “pharmaceutical composition” is intended to include the combination of an active agent with a carrier, inert or active, making the composition suitable for diagnostic or therapeutic use in vitro, in vivo or ex vivo.

As used herein, the term “pharmaceutically acceptable carrier” encompasses any of the standard pharmaceutical carriers, such as a phosphate buffered saline solution, water, and emulsions, such as an oil/water or water/oil emulsion, and various types of wetting agents. The compositions also can include stabilizers and preservatives. For examples of carriers, stabilizers and adjuvants, see Martin, REMINGTON'S PHARM. SCI., 15th Ed. (Mack Publ. Co., Easton (1975)).

An “effective amount” is an amount sufficient to effect beneficial or desired results.

An effective amount can be administered in one or more administrations, applications or dosages.

A “subject,” “individual” or “patient” is used interchangeably herein, which refers to a vertebrate, preferably a mammal, more preferably a human. Mammals include, but are not limited to, murines, simians, humans, farm animals, sport animals, and pets.

A “control” is an alternative subject or sample used in an experiment for comparison purpose. A control can be “positive” or “negative”. For example, where the purpose of the experiment is to determine a correlation of an altered expression level of a gene with a particular type of cancer, it is generally preferable to use a positive control (a subject or a sample from a subject, carrying such alteration and exhibiting syndromes characteristic of that disease), and a negative control (a subject or a sample from a subject lacking the altered expression and clinical syndrome of that disease).

A “gene delivery vehicle” is defined as any molecule that can carry inserted polynucleotides into a host cell. Examples of gene delivery vehicles are liposomes, cationic liposomes, viruses, such as baculovirus, adenovirus, adeno-associated virus, and retrovirus, bacteriophage, cosmid, plasmid, fungal vectors and other recombination vehicles typically used in the art which have been described for expression in a variety of eukaryotic and prokaryotic hosts, and may be used for gene therapy as well as for simple protein expression.

A “viral vector” is defined as a recombinantly produced virus or viral particle that comprises a polynucleotide to be delivered into a host cell, either in vivo, ex vivo or in vitro. Examples of viral vectors include retroviral vectors, adenovirus vectors, adeno-associated virus vectors and the like. In aspects where gene transfer is mediated by a retroviral vector, a vector construct refers to the polynucleotide comprising the retroviral genome or part thereof, and the inserted polynucleotide. As used herein, “retroviral mediated gene transfer” or “retroviral transduction” carries the same meaning and refers to the process by which a gene or nucleic acid sequences are stably transferred into the host cell by virtue of the virus entering the cell and integrating its genome into the host cell genome. The virus can enter the host cell via its normal mechanism of infection or be modified such that it binds to a different host cell surface receptor or ligand to enter the cell. As used herein, retroviral vector refers to a viral particle capable of introducing exogenous nucleic acid into a cell through a viral or viral-like entry mechanism.

Retroviruses carry their genetic information in the form of RNA; however, once the virus infects a cell, the RNA is reverse-transcribed into the DNA form which integrates into the genomic DNA of the infected cell. The integrated DNA form is called a provirus.

In aspects where gene transfer is mediated by a DNA viral vector, such as an adenovirus (Ad) or adeno-associated virus (AAV), a vector construct refers to the polynucleotide comprising the viral genome or part thereof, and a polynucleotide to be inserted. Adenoviruses (Ads) are a relatively well characterized, homogenous group of viruses, including over 50 serotypes. (see, e.g., WO 95/27071). Ads are easy to grow and do not require integration into the host cell genome. Recombinant Ad-derived vectors, particularly those that reduce the potential for recombination and generation of wild-type virus, have also been constructed. (see, WO 95/00655; WO 95/11984). Wild-type AAV has high infectivity and specificity integrating into the host cells genome. (Hermonat and Muzyczka (1984) PNAS USA 81:6466-6470; Lebkowski, et al. (1988) Mol. Cell. Biol. 8:3988-3996).

Vectors that contain both a promoter and a cloning site into which a polynucleotide can be operatively linked are well known in the art. Such vectors are capable of transcribing RNA in vitro or in vivo, and are commercially available from sources such as Stratagene (La Jolla, Calif.) and Promega Biotech (Madison, Wis.). In order to optimize expression and/or in vitro transcription, it may be necessary to remove, add or alter 5′ and/or 3′ untranslated portions of the clones to eliminate extra, potential inappropriate alternative translation initiation codons or other sequences that may interfere with or reduce expression, either at the level of transcription or translation. Alternatively, consensus ribosome binding sites can be inserted immediately 5′ of the start codon to enhance expression.

Gene delivery vehicles also include several non-viral vectors, including DNA/liposome complexes, and targeted viral protein DNA complexes. Liposomes that also comprise a targeting antibody or fragment thereof can be used in the methods of this invention. To enhance delivery to a cell, the nucleic acid or proteins of this invention can be conjugated to antibodies or binding fragments thereof which bind cell surface antigens, e.g., TCR, CD3 or CD4.

Polynucleotides are inserted into vector genomes using methods well known in the art. For example, insert and vector DNA can be contacted, under suitable conditions, with a restriction enzyme to create complementary ends on each molecule that can pair with each other and be joined together with a ligase. Alternatively, synthetic nucleic acid linkers can be ligated to the termini of restricted polynucleotide. These synthetic linkers contain nucleic acid sequences that correspond to a particular restriction site in the vector DNA. Additionally, an oligonucleotide containing a termination codon and an appropriate restriction site can be ligated for insertion into a vector containing, for example, some or all of the following: a selectable marker gene, such as the neomycin gene for selection of stable or transient transfectants in mammalian cells; enhancer/promoter sequences from the immediate early gene of human CMV for high levels of transcription; transcription termination and RNA processing signals from SV40 for mRNA stability; SV40 polyoma origins of replication and ColE1 for proper episomal replication; versatile multiple cloning sites; stabilizing elements3′ to the inserted polynucleotide, and T7 and SP6 RNA promoters for in vitro transcription of sense and antisense RNA. Other means are well known and available in the art.

“Host cell” is intended to include any individual cell or cell culture which can be or have been recipients for vectors or the incorporation of exogenous polynucleotides, polypeptides and/or proteins. It also is intended to include progeny of a single cell, and the progeny may not necessarily be completely identical (in morphology or in genomic or total DNA complement) to the original parent cell due to natural, accidental, or deliberate mutation. The cells may be prokaryotic or eukaryotic, and include but are not limited to bacterial cells, yeast cells, plant cells, insect cells, animal cells, and mammalian cells, e.g., murine, rat, simian or human.

An “antibody” is an immunoglobulin molecule capable of binding an antigen. As used herein, the term encompasses not only intact immunoglobulin molecules, but also anti-idiotypic antibodies, mutants, fragments, fusion proteins, humanized proteins and modifications of the immunoglobulin molecule that comprise an antigen recognition site of the required specificity.

As used herein, “expression” refers to the process by which polynucleotides are transcribed into mRNA and translated into peptides, polypeptides, or proteins. If the polynucleotide is derived from genomic DNA, expression may include splicing of the mRNA, if an appropriate eukaryotic host is selected. Regulatory elements required for expression include promoter sequences to bind RNA polymerase and transcription initiation sequences for ribosome binding. For example, a bacterial expression vector includes a promoter such as the lac promoter and for transcription initiation the Shine-Dalgarno sequence and the start codon AUG (Sambrook, et al. (1989) supra). Similarly, an eukaryotic expression vector includes a heterologous or homologous promoter for RNA polymerase II, a downstream polyadenylation signal, the start codon AUG, and a termination codon for detachment of the ribosome. Such vectors can be obtained commercially or assembled by the sequences described in methods well known in the art, for example, the methods described below for constructing vectors in general.

A “subject” is a vertebrate, preferably a mammal, more preferably a human. Mammals include, but are not limited to, murines, simians, humans, farm animals, sport animals, and pets.

A “control” is an alternative subject or sample used in an experiment for comparison purpose. A control can be “positive” or “negative.”

A “candidate agent” suitable for assaying in the methods of the subject application may be any type of molecule from, for example, chemical, nutritional or biological sources. The agent may be a naturally occurring or synthetically produced. For example, the agent may encompass numerous chemical classes, though typically they are organic molecule, preferably small organic compounds having a molecular weight of more than 50 and less than about 2,500 Daltons. Such molecules may comprise functional groups necessary for structural interaction with proteins or nucleic acids. By way of example, chemical agents may be novel, untested chemicals, agonists, antagonists, or modifications of known therapeutic agents.

Candidate agents may also be found among biomolecules including, but not limited to, peptides, saccharides, fatty acids, antibodies, steroids, purines, pryimidines, toxins conjugated cytokines, derivatives or structural analogs thereof or a molecule manufactured to mimic the effect of a biological response modifier. Examples of candidate agents from nutritional sources include, but is not limited to, extracts from plant or animal sources or extracts thereof.

Candidate agents may be obtained from a wide variety of sources including libraries of synthetic or natural compounds. Alternatively, libraries of natural compounds in the form of bacterial, fungal, plant, and animal extracts are available or readily produced, natural or synthetically produced libraries or compounds are readily modified through conventional chemical, physical and biochemical means, and may be used to produce combinatorial libraries. Known pharmacological agents may be subjected to random or directed chemical modifications, such as acylation, alkylation, esterification, amidification, etc. to produce structural analog

A ligand may be any protein or portions of a protein that may interact with the FRP-4 gene product or protein. Such ligand or ligands may be soluble or membrane bound. The ligand or ligands may be a naturally occurring protein, or synthetically or recombinantly produced. The ligand may also be a nonprotein molecule(e.g., small molecule) that acts as ligand when it interacts with the FRP-4 protein or a molecule described herein above for candidate agents. Interactions between the ligand and ligand binding domain of FRP-4 include, but are not limited to, any covalent or non-covalent interactions. The ligand binding domain may be any region of the FRP-4 molecule that interacts directly or indirectly with the FRP-4 ligand.

As used herein, the term “cytokine” refers to any one of the numerous factors that exert a variety of effects on cells, for example, inducing growth or proliferation. Non-limiting examples of cytokines which may be used alone or in combination in the practice of the present invention include, interleukin-2 (IL-2), stem cell factor (SCF), interleukin 3 (IL-3), interleukin 6 (IL-6), interleukin 12 (IL-12), G-CSF, granulocyte macrophage-colony stimulating factor (GM-CSF), interleukin-1 alpha (IL-1), interleukin-11 (IL-11), MIP-1, leukemia inhibitory factor (LIF), c-kit ligand, thrombopoietin (TPO) and flt3 ligand. The present invention also includes culture conditions in which one or more cytokine is specifically excluded from the medium. Cytokines are commercially available from several vendors such as, for example, Genentech (South San Francisco, Calif.), Amgen (Thousand Oaks, Calif.), R&D Systems (Minneapolis, Minn.) and Immunex (Seattle, Wash.). It is intended, although not always explicitly stated, that molecules having similar biological activity as wild-type or purified cytokines (e.g., recombinantly produced or muteins thereof) are intended to be used within the spirit and scope of the invention.

The term “culturing” refers to the in vitro propagation of cells or organisms on or in media of various kinds. It is understood that the descendants of a cell grown in culture may not be completely identical (i.e., morphologically, genetically, or phenotypically) to the parent cell. By “expanded” is meant any proliferation or division of cells.

The terms “cancer,” “neoplasm,” and “tumor,” used interchangeably and in either the singular or plural form, refer to cells that have undergone a malignant transformation that makes them pathological to the host organism. Primary cancer cells (that is, cells obtained from near the site of malignant transformation) can be readily distinguished from non-cancerous cells by well-established techniques, particularly histological examination. The definition of a cancer cell, as used herein, includes not only a primary cancer cell, but any cell derived from a cancer cell ancestor. This includes metastasized cancer cells, and in vitro cultures and cell lines derived from cancer cells. When referring to a type of cancer that normally manifests as a solid tumor, a “clinically detectable” tumor is one that is detectable on the basis of tumor mass; e.g., by such procedures as CAT scan, magnetic resonance imaging (MRI), X-ray, ultrasound or palpation. Biochemical or immunologic findings alone may be insufficient to meet this definition. Tumor cells often express antigens which are tumor specific. The term “tumor associated antigen” or “TAA” refers to an antigen that is associated with or specific to a tumor.

As used herein, “solid phase support” is not limited to a specific type of support. Rather a large number of supports are available and are known to one of ordinary skill in the art. Solid phase supports include silica gels, resins, derivatized plastic films, glass beads, cotton, plastic beads, alumina gels. A suitable solid phase support may be selected on the basis of desired end use and suitability for various synthetic protocols. For example, for peptide synthesis, solid phase support may refer to resins such as polystyrene (e.g., PAM-resin obtained from Bachem Inc., Peninsula Laboratories, etc.), POLYHIPE® resin (obtained from Aminotech, Canada), polyamide resin (obtained from Peninsula Laboratories), polystyrene resin grafted with polyethylene glycol (TentaGel™, Rapp Polymere, Tubingen, Germany) or polydimethylacrylamide resin (obtained from Milligen/Biosearch, California). In a preferred embodiment for peptide synthesis, solid phase support refers to polydimethylacrylamide resin.

A “transgenic animal” refers to a genetically engineered animal or offspring of genetically engineered animals. The transgenic animal may contain genetic material from at least one unrelated organism (such as from a bacteria, virus, plant, or other animal) or may contain a mutation which interferes with expression of a gene product.

The term “oncogenic osteomalacia” (OOM), “oncogenic hypophosphatemic osteomalacia” (OHO), or “tumor-associated osteomalacia” refers to a tumor-acquired syndrome characterized mainly by hypophosphatemia, hyperphosphaturia, abnormally low serum level of 1,25-dihydroxyvitamin D, and osteomalacia. Tumors associated with OOM are mainly of mesenchymal origin such as hemangiopericytomas, although carcinoma of prostate and lung, fibrous dysplasia of bone, linear sebaceous naevus syndrome, neurofibromatosis, and oat cell carcinoma are also associated with OOM. Thus, the OOM syndrome can be described as having a paraneoplastic etiology. Surgical removal of the tumor in a patient often results in a complete or near-complete resolution of biochemical and clinical defects associated with OOM.

The terms “phosphatonin” and “phosphatonin-related” are used interchangeably to refer to a polypeptide humoral factor specifically involved in the regulation of phosphate homeostasis. A factor with “phosphatonin activity” down regulates the renal re-absorption of inorganic phosphate. Phosphatonin activity incorporates the combined function of phosphatonin and additional modulating factors.

Oncogenic osteomalacia-related genes include genes that have been identified to be over-expressed or under-expressed relative to control tumors (histologically similar tumors that are not associated with OOM). Genes that are up-regulated or down-regulated in oncogenic osteomalacia may encode proteins involved in several distinct biochemical pathways. These include phosphate regulation, bone mineralization, and protein synthesis, processing and secretion.

The regulation of phosphate metabolism plays a central role in mediating the symptoms of oncogenic osteomalacia. Genes whose expression is altered in OOM tumors can effect phosphate metabolism through a variety of mechanisms. For example, the tumor may directly produce increased amounts of phosphatonin, a secreted humoral factor whose activity includes inhibition of phosphate re-absorption in the kidney. Alternatively the OOM tumor cells could produce a factor or factors that alter the expression in the kidney of accessory polypeptides required for mediating the effects of phosphatonin such as the phosphatonin receptor and intracellular proteins responsible for eliciting the effects of phosphatonin.

Altered gene expression by OOM tumor cells can also alter phosphate metabolism by more complex mechanisms. For example tumor produced factors could up-regulate expression of genes normally controlled directly by phosphatonin or in response to phosphatonin. Such OOM tumor produced factors could increase expression of phosphate transport molecules and other cellular proteins necessary for regulating either phosphate uptake or secretion of phosphate. Alternatively, OOM tumor factors could alter expression of extracellular regulators or carriers of phosphate or phosphatonin.

OOM-related genes that modulate phosphate metabolism are useful candidates for developing therapeutic agents for a variety of disease conditions related to abnormal phosphate metabolism. These include renal conditions such as renal osteodystrophy, changes in phosphate homeostasis after kidney transplant, end stage renal disease (ESRD), and acute renal disease, bone defects, hypophosphatasemia, hyperphosphatasemia, hypoparathyroidism, and pseudohypoparathyroidism.

Phosphate metabolism related factors could provide useful mediators of disease conditions through a variety of alternative mechanisms. For example, during ESRD, phosphatonin or other proteins in its pathway may inhibit absorption of phosphate in the small intestine. Such factors may also enhance phosphate uptake in the proximal tubules of the kidney. Modulation of the activity of these factors could therefore be used to control the symptoms of this disease.

In conditions characterized by hypophosphatemia or low serum phosphate levels blocking any protein that is involved in lowering serum phosphate levels or inhibiting its functions could be an effective therapy. This type of therapy could be useful for a range of conditions including hyperparathyroidism, X-linked hypophosphatemic rickets, vitamin D dependent rickets, Franconi Syndrome, post kidney transplant condition, and oncogenic osteomalacia.

Diseases characterized by increased phosphate levels or hyperphosphatemia could be affected by treatment directed towards any protein that acts in the phosphatonin pathway to lower serum phosphate levels. Diseases related to hyperphosphatemia include: hypoparathyroidism (levels of PTH secreted are insufficient to maintain extracellular calcium and phosphate levels-leads to hypocalcemia and hyperphosphatemia); pseudohypoparathyroidism (a group of disorders characterized by biochemical hypoparathyroidism, hypocalcemia and hyperphosphatemia, increased secretion of PTH and resistance to the biological actions of PTH); transcellular phosphate shift from cells into the extracellular fluid caused by systemic infections, severe hyperthermia, crush injuries, non-traumatic rhabdomyolysis, and tumor lysis syndrome after cytotoxic therapies for hematologic malignancies; and renal disease.

In addition to modulation of phosphate metabolism, factors whose expression is altered in OOM tumor cells can include genes whose polypeptide products act directly on osteogenic cells to mediate bone mineralization. Such proteins associated with OOM may either promote or inhibit diseases associated with defective mineralization. Possible functions of proteins in the bone mineralization pathway include: inhibition of bone mineralization, regulation of the early stages of bone mineralization, and control of bone cell differentiation and bone development.

A variety of types of polypeptide factors may be found to modulate bone mineralization. For example extracellular matrix proteins (ECM) are an important constituent of bone. In bone, cartilage and the tissues forming the teeth, unlike those in other connective tissues, the matrices have the unique ability to become calcified. Furthermore, control of cell viability and morphogenesis is well known to be affected by appropriate contact with a wide array of ECM proteins. Thus OOM tumor produced ECM proteins could alter the natural process of bone mineral homeostasis by acting directly on bone cells. Alternatively, OOM tumor cells could produce diffuisable soluble factors that regulate bone cell differentiation, growth and metabolism. Such factors also provide useful targets for development of therapeutic agents to regulate bone mineralization.

A number of serious pathological conditions are related to defects in bone mineralization. These include osteoporosis (a metabolic bone disease characterized by low bone mass and micro architectural deterioration of bone tissue); osteomalacia (a defect in bone mineralization that occurs after the cessation of growth and involves only the bone and not the growth plate); rickets (a disorder of mineralization of the bone matrix, or osteoid, in growing bones; that involves both the growth plate (epiphysis) and newly formed traebacular and cortical bone); hypophosphatasias (a rare heritable type of rickets or osteomalacia (1 in 100,000 births) characterized by a reduction of activity of the tissue nonspecific isoenzyme of alkaline phosphatase); and Fanconi syndrome and renal tubular acidosis (a generalized defect in renal proximal tubule transport capacity that includes impaired reabsorption of glucose, phosphate, amino acids, bicarbonate, uric acid, citrate and other organic acids, and low-molecular weight proteins and that is associated with rickets and osteomalacia).

OOM tumor produced factors that are found to modulate fundamental processes involved in bone formation, mineralization and maintenance could provide useful targets to inhibit the progression of these diseases.

In addition to diseases characterized by defects in bone mineralization, pathological conditions of the bone include defects in bone remodeling such as Paget's disease, osteomyeloitis, osteosarcoma and stress fracture. As in the case of defective bone mineralization, polypeptide factors identified from OOM tumor cells that directly modulate bone metabolism and bone cell development are useful targets for developing novel therapeutic agents to treat diseases characterized by alternative bone pathologies. Furthermore, in certain cases, expression of OOM tumor associated factors may be found to be diagnostic of bone disease making these genes useful markers for diagnostic tests to identify such conditions.

Therapeutic Applications

The present invention provides methods of modulating phosphate homeostasis and/or bone mineralization in a subject by altering the activity of the FRP-4 gene or by altering the activity of the FRP-4 protein. In one embodiment of the invention phosphate homeostasis is modulated by delivering to a subject an effective amount of an agent that alters the activity of FRP-4. Agents that are useful for practicing the invention include, but are not limited to small organic molecules, polypeptides and antibodies. Agents that inhibit the activity of FRP-4 protein are useful for increasing phosphate re-absorption while agents that stimulate FRP-4 activity are useful for decreasing phosphate re-absorption. Thus, the methods of the invention can be used for conditions characterized by either hypophosphatemia or hyperphosphatemia.

In a separate embodiment of the invention phosphate homeostasis is modulated by delivering an agent that alters the expression of the FRP-4 gene. Such agents can include, but are not limited to, gene delivery vehicles comprising anti-sense RNA molecules and ribozymes, anti-sense oligonucleotides, and small organic molecules or polypeptides that specifically inhibit expression of the FRP-4 gene. Inhibition of expression of the FRP-4 gene is useful to increase the re-absorption of phosphate in a subject.

The invention also provides methods of modulating renal phosphate transport by delivering an agent that alters FRP-4 protein activity and/or by delivering an agent that alters FRP-4 gene expression. In addition, the present invention encompasses methods of alleviating the symptoms of hypophosphatemia or hyperphosphatemia related diseases, such as oncogenic osteomalacia. To perform the methods of the invention, polynucleotides, polypeptides and therapeutic agents and their derivatives can be used, either alone or in conjunction with other active agents, in a pharmaceutical composition for the therapeutic treatments described herein. Symptoms that can be alleviated include, but are not limited to hypophosphatemia, phosphaturia, low serum concentration of 1,25-dihydroxyvitamin D and osteomalacia.

In one embodiment, a pharmaceutical composition comprising an agent identified by the screening assay described below is administered to a subject in an effective amount to treat hypophosphatemia related diseases, such as oncogenic osteomalacia, or to ameliorate the symptoms associated therewith. Preferably, the pharmaceutical composition is capable of modulating FRP-4 protein function in a subject with oncogenic osteomalacia; and thereby restoring normal serum phosphate levels in the subject.

Delivery of FRP-4 gene and protein modulating agents is useful for treatment of phosphate homeostasis related diseases that include X-linked hypophosphatemia rickets, oncogenic osteomalacia, rhabdomyolysis, cardiomyopathy, tumoral calcinosis, renal failure and bone mineralization.

In another embodiment, a pharmaceutical composition comprising an FRP-4 polypeptide is administered to a subject in an effective amount to reduce phosphate reabsorption. Preferably, the pharmaceutical composition contains FRP-4 polypeptide, a secreted protein, and is capable of lowering the abnormally elevated serum phosphate levels in patients with phosphate homeostasis-related disease. Alternatively, the FRP-4 polypeptide-containing pharmaceutical composition further comprises active agents that promote the desired function in regulating phosphate homeostasis. Suitable active agents include, but are not limited to, enzymes or co-factors that are involved in the posttranslational modification and processing of the mature FRP-4 protein; or factors lo responsible for maintaining the activated form of FRP-4 polypeptide in circulation and at the site of phosphate homeostasis.

Alternatively, anti FRP-4 ligand antibodies can be induced by administering anti-idiotype antibodies as immunogens. By way of example, an antibody preparation prepared as described herein may be used to induce anti-idiotype antibody in a host animal. The composition is administered to the host animal in a suitable diluent. Following administration, usually repeated administration, the host produces anti-idiotype antibody. To eliminate an immunogenic response to the Fc region, antibodies produced by the same species as the host animal can be used or the Fc region of the administered antibodies can be removed. Following induction of anti-idiotype antibody in the host animal, serum or plasma is removed to provide an antibody composition. The composition can be purified by methods known in the art (e.g., affinity chromatography).

Various delivery systems are known and can be used to administer a therapeutic agent, e.g., encapsulation in liposomes, microparticles, microcapsules, expression by recombinant cells, receptor-mediated endocytosis (see, e.g., Wu and Wu (1987) J. Biol. Chem. 262:4429-4432), construction of a therapeutic nucleic acid as part of a retroviral or other vector, etc. Methods of delivery include but are not limited to transdermally, gene therapy, intra-arterial, intra-muscular, intravenous, intranasal, and oral routes, and include sustained delivery systems. In a specific embodiment, it may be desirable to administer the pharmaceutical compositions of the invention locally to the area in need of treatment; this may be achieved by, for example, and not by way of limitation, local infusion during surgery, by injection, or by means of a catheter or targeted gene delivery of the sequence coding for the therapeutic.

The pharmaceutical compositions identified herein as effective for their intended purpose can be administered to subjects or individuals susceptible to or at risk of developing diseases associated with abnormal phosphate transport in the kidney. When the agent is administered to a subject such as a mouse, a rat or a human patient, the agent can be added to a pharmaceutically acceptable carrier and systemically or topically administered to the subject. Therapeutic amounts can be empirically determined and will vary with the pathology being treated, the subject being treated and the efficacy and toxicity of the agent.

Administration in vivo can be effected in one dose, continuously or intermittently throughout the course of treatment. Methods of determining the most effective means and dosage of administration are well known to those of skill in the art and will vary with the composition used for therapy, the purpose of the therapy, the target cell being treated, and the subject being treated. Single or multiple administrations can be carried out with the dose level and pattern being selected by the treating physician. Suitable dosage formulations and methods of administering the agents can be found below.

The agents and compositions useful for practicing the methods of the present invention can be used in the manufacture of medicaments and for the treatment of humans and other animals by administration in accordance with conventional procedures, such as an active ingredient in pharmaceutical compositions. The pharmaceutical compositions can be administered orally, intranasally, parenterally, transdermally or by inhalation therapy, and may take the form of tablets, lozenges, granules, capsules, pills, ampoules, suppositories or aerosol form. They may also take the form of gene therapy, suspensions, solutions and emulsions of the active ingredient in aqueous or nonaqueous diluents, syrups, granulates or powders. In addition to an agent of the present invention, the pharmaceutical compositions can be combined with other therapeutically useful agents.

Expression Analysis

The present invention also provides methods for detecting a cell expressing a polypeptide encoded by the FRP-4 gene by contacting a suitable sample with a suitable polynucleotide probe under conditions of moderate hybridization stringency and detecting any complementary nucleotides, thereby detecting the cell.

A suitable polynucleotide probe can be derived from the sequence of the FRP-4 cDNA shown in SE ID NO 1 by preparing an oligonucleotide or polynucleotide molecule that is complementary to a portion of the FRP-4 cDNA. An oligonucleotide probe ranging in size from 10 or 20 nucleotides to about 50 nucleotides can be produced using an automated DNA synthesizer. Alternatively, polynucleotide probes can be prepared from an isolated polynucleotide that comprises the FRP-4 cDNA sequence using methods well known in the art such as PCR or nick translation using various DNA polymerase enzymes. Such probes typically range in size from 50 to 500 base pairs in length. Suitable polynucleotide probes should not contain repeated DNA motifs and should not have high homology to genes other than the target FRP-4 sequence.

The invention further provides a method of detecting a cell expressing polypeptide encoded by the FRP-4 gene by performing RT-PCR on a suitable sample using a primer pair derived from the FRP-4 cDNA sequence. RT-PCR can be performed using methods well established in the art. A suitable primer pair will be oligonucleotides of similar annealing temperatures that are complementary to sequences on opposite strands of the FRP-4 cDNA. The primers should amplify a portion of the FRP-4 cDNA ranging from 50 to 1,000 base pairs, preferably 250 to 750 base pairs in length. Optimal conditions for performing PCR can be determined without undue experimentation by comparing a series of alternative reaction conditions in which reaction conditions such as primer concentration, magnesium concentration, annealing temperature and cycle number are varied, to identify appropriate PCR conditions.

Methods of detecting and monitoring FRP-4 expression are useful for detecting a neoplastic cell associated with oncogenic osteomalacia. A suitable sample for such analysis can be obtained from a tissue sample removed from subject. When practiced in vivo, the methods are useful for localizing an osteogenic osteomalacia inducing tumor. The methods of detecting FRP-4 expression levels can be used to quantitate FRP-4 expression levels. Furthermore, it is useful to compare the level of FRP-4 expression in normal and diseased cells to determine levels of expression that are indicative of abnormal phosphate metabolism. Thus the present invention envisions using these methods to identify subjects that are appropriate candidates for treatment using the methods of this invention. Finally, the methods of detecting FRP-4 gene expression are also useful for monitoring a gene delivery vehicle comprising the FRP-4 gene sequence when such a gene delivery vehicle is administered to a subject.

The above methods can be further modified by use of an activated cell, defined above.

Modulating the Phenotype of a Cell

The present invention further provides a method for modulating the phenotype of a neoplastic cell associated with oncogenic osteomalacia comprising delivering an agent that alters the expression of a polynucleotide encoding the FRP-4 polypeptide. Appropriate subjects for receiving such an agent can be identified by performing the FRP-4 gene expression analysis methods described above.

In addition, the invention provide methods for modulating the phenotype of a cell associated with phosphate homeostasis comprising delivering an agent that alters the expression of the FRP-4 gene. This method is useful for modulating FRP-4 expression and phosphate homeostasis in a subject. Agents that enhance the expression of FRP-4 are useful for reducing the re-absorption of phosphate in the kidney while agents that inhibit the expression of FRP-4 are useful for increasing phosphate re-absorption.

Screening Assays for Candidate Agents

The present invention provides methods for screening various agents that modulate the expression of the FRP-4 gene or the activity of the FRP-4 protein. These agents are useful for modulating phosphate homeostasis in a subject, for modulating renal phosphate transport, or alleviating the symptoms associated with oncogenic osteomalacia, for treating phosphate homeostasis-related disease and for altering the phenotype of a neoplastic cell associated with oncogenic osteomalacia or a cell associated with phosphate homeostasis or bone mineralization. For the purposes of this invention, an “agent” is intended to include, but not be limited to a biological or chemical compound such as a simple or complex organic or inorganic molecule, a peptide, a protein (e.g. antibody), a polynucleotide (e.g. anti-sense) or a ribozyme. A vast array of compounds can be synthesized, for example polymers, such as polypeptides and polynucleotides, and synthetic organic compounds based on various core structures, and these are also included in the term “agent”. In addition, various natural sources can provide compounds for screening, such as plant or animal extracts, and the like. It should be understood, although not always explicitly stated that the agent is used alone or in combination with another agent, having the same or different biological activity as the agents identified by the inventive screen.

One preferred embodiment is a method for screening small molecules capable of interacting with the FRP-4 polypeptide produced from a neoplastic cell associated with oncogenic osteomalacia. For the purpose of this invention, “small molecules” are molecules having low molecular weights (MW) that are, in one embodiment, capable of binding to a protein of interest such as FRP-4 polypeptide, and thereby altering the function of the protein. Preferably, the MW of a small molecule is no more than1,000. Methods for screening small molecules capable of altering protein function are known in the art. For example, a miniaturized arrayed assay for detecting small molecule-protein interactions in cells is discussed by You et al. (1997) Chem. Biol. 4:961-968.

To practice the screening method in vitro, suitable cell cultures or tissue cultures containing this type of neoplastic cell are first provided. The cell can be a cultured cell or a genetically modified cell in which FRP-4, or its complement is expressed. Alternatively, the cells can be from a tissue biopsy. The cells are cultured under conditions (temperature, growth or culture medium and gas (CO₂)) and for an appropriate amount of time to attain exponential proliferation without density dependent constraints. It also is desirable to maintain an additional separate cell culture; one which does not receive the agent being tested as a control.

As is apparent to one of skill in the art, suitable cells may be cultured in microtiter plates and several agents may be assayed at the same time by noting genotypic changes, phenotypic changes or cell death.

When the agent is a composition other than a DNA or RNA, such as a small molecule as described above, the agent may be directly added to the cell culture or added to culture medium for addition. As is apparent to those skilled in the art, an “effective” amount must be added which can be empirically determined. When the agent is a polynucleotide, it may be directly added by use of a gene gun or electroporation. Alternatively, it may be inserted into the cell using a gene delivery vehicle or other method as described herein.

The invention also provides screening assays for agents having the ability to compete with a FRP-4 ligand. A number of competitive binding assays are known in the art. Generally, competitive binding assays rely on the ability of a labeled standard to compete with the candidate agent for binding with a limited amount of ligand. Examples of competitive assay systems include, but are not limited to, radioimmunoassays (RIA), enzyme immunoassays (EIA), preferably the enzyme linked immunosorbent assay (ELISA), “sandwich” immunoassays, immunoradiometric assays, fluorescent immunoassays, and immunoelectrophoresis.

Generally, in such assays the ligand will be labeled with a detectable moiety (the detectably labeled ligand hereafter called the “tracer”) and used in a competition assay with a candidate agent for binding the FRP-4 ligand domain. Numerous detectable labels are available including, but not limited to, radioisotopes (e.g., ³⁵S, ¹⁴C, ¹²⁵I, ³H, and ¹³¹I, see also Coligen et al., eds., Current Protocols in Immunology, Volumes 1 and 2 (1991), Wiley-Interscience, New York, N.Y.); fluorescent labels (e.g., rare earth chelates (europium chelates), fluorescein and its derivatives, rhodamine and its derivatives, dansyl, lissamine, phycoerythrin and Texas Red are available, see also Current Protocols in Immunology); enzyme-substrate labels (e.g., U.S. Pat. No. 4,275,149); and enzyme-substrate combinations (e.g., horseradish peroxidase (HRP) with hydrogen peroxidase as a substrate; alkaline phosphatase (AP) with para-nitrophenyl phosphate as chromogenic substrate, β-D-galactosidase (β-D-Gal) with a chromogenic substrate ). In such assays, the tracer is generally incubated with, the ligand binding domain or a biologically active portion of FRP-4 in the presence of varying concentrations of unlabeled candidate agent. Increasing concentrations of successful candidate compound effectively compete with binding of the tracer to FRP-4 (Goodman &Gilman's, (9^{th} Edition)“The Pharmacological Basis of Therapeutics”, 1996; B. C. Cunningham, D. G. Lowe, B. Li, B. D. Bennett, and J. A. Wells, EMBO J. 13:2508 (1994)).

The assays also can be performed in a subject. When the subject is an animal such as a rat, mouse or simian, the method provides a convenient animal model system which can be used prior to clinical testing of an agent. In this system, a candidate agent is a potential drug if transcript expression is altered, i.e., upregulated (such as restoring tumor suppressor function), downregulated or eliminated as with drug resistant genes or oncogenes, or if symptoms associated or correlated to the presence of cells containing transcript expression are ameliorated, each as compared to untreated, animal having the pathological cells. It also can be useful to have a separate negative control group of cells or animals which are healthy and not treated, which provides a basis for comparison. After administration of the agent to subject, suitable cells or tissue samples are collected and assayed for altered gene expression or protein function.

Kits containing the agents and instructions necessary to perform the screen and in vitro or in vivo methods as described herein also are claimed.

The invention further pertains to novel agents identified by the screening assays described herein and uses thereof for treatments as described, supra.

Screening Assays for FRP-4 Ligand

The invention also provides aa assay for screening for ligand that modulate the activity of FRP-4 protein activity. Ano of the screeing assays described herein above may be used to screen for ligands. Candidate ligands may be obtained from the same souces as for candidate agents.

In one embodiment, the invention provides assays for screening a ligand which bind to or modulate the activity of the membrane bound form of an FRP-4 protein or polypeptide or biologically-active portion thereof. By way of example, a cell based assay may be used. In a cell-based assay, a cell (e.g., mammalian, yeast etc) which expresses a membrane-bound form of FRP-4 protein, or a biologically-active portion thereof, on the cell surface is contacted with a candidate ligand and the ability of the candidate ligand to bind to FRP-4 is determined. Determining the ability of the test compound to bind to the FRP-4 protein can be determined either directly or indirectly. For example, the candidate ligand may be coupled with a detectable label (e.g., radioisotope, enzymatic label) so that binding can be determined by detecting the labeled compound in a complex. In an alternative embodiment, determining the ability of the test compound to modulate the activity of FRP-4 protein can be accomplished by determining the ability of the FRP-4 protein to modulate an FRP-4 target molecule.

In still another embodiment, an assay is a cell-free assay comprising contacting FRP-4 protein or biologically-active portion thereof with a test compound and determining the ability of the test compound to modulate (e.g. stimulate or inhibit) the activity of the FRP-4 protein or biologically-active portion thereof. The cell-free assays of the invention are amenable to use of both the soluble form or the membrane-bound form of FRP-4 protein. In the case of cell-free assays comprising the membrane-bound form of FRP-4 protein, it may be desirable to utilize a solubilizing agent (e.g., non-ionic detergents)such that the membrane-bound form of FRP-4 protein is maintained in solution.

In more than one embodiment of the above assay methods of the invention, it may be desirable to immobilize either FRP-4 protein or candidate ligand on a substrate by methods known in the art.

In yet another aspect of the invention, the FRP-4 proteins can be used as “bait proteins” in a two-hybrid assay or three hybrid assay (see, e.g., U.S. Pat. No. 5,283,317; Zervos, et al., 1993. Cell 72: 223-232; Madura, et al., 1993. J Biol. Chem. 268: 12046-12054; Bartel, et al., 1993. Biotechniques 14: 920-924; Iwabuchi, et al., 1993. Oncogene 8: 1693-1696; and Brent WO 94/10300), to identify other proteins that bind to or interact with FRP-4 (“FRP-4-binding proteins” or “FRP-4-bp”) and modulate FRP-4 activity. Such FRP-4-binding proteins are also likely to be involved in the propagation of signals by the FRP-4 proteins as, for example, upstream or downstream elements of the FRP-4 pathway.

Polynucleotides

An isolated polynucleotide encoding FRP-4 is provided by this invention. Polynucleotides comprising the sequence of the FRP-4 gene are useful for practicing various embodiments of the present invention. In a further aspect, the polynucleotide also comprises a sequence. These polynucleotides include, but are not limited to probes for detecting and monitoring gene expression, primers for performing polymerase chain reaction (PCR), cDNA molecules encoding FRP-4 polypeptide, gene delivery vehicles to deliver FRP-4 polynucleotides to a cell, expression vectors for the production of FRP-4 protein, and anti-sense polynucleotides and ribozymes to modulate FRP-4 expression. The sequence of a cDNA comprising the human FRP-4 cDNA is provided in the Figure (SEQ ID NO. 1). One of skill in the art will be familiar with a variety of means by which to detect and obtain such an isolated polynucleotide. Descriptions of several of these methods are provided below.

In addition to the sequence shown in SEQ ID NO. 1 the methods of this invention can be practiced using anti-sense polynucleotides, e.g. antisense RNA, complementary to this sequence. One can obtain an antisense RNA using the sequence provided in SEQ ID NO. 1, and the methodology described in Vander Krol, et al. (1988) BioTechniques 6:958.

The polynucleotides can be introduced by any suitable gene delivery method or vector. They also can be expressed in a suitable host cell for generating a cell-based therapy. These methods are described in more detail below.

This invention can also utilize genetically modified cells that produce enhanced expression of FRP-4 polypeptide as compared to wild-type cells. The genetically modified cells can be produced by insertion of upstream regulatory sequences such as promoters or gene activators (see U.S. Pat. No. 5,733,761).

The polynucleotides and sequences identified above can be conjugated to a detectable marker, e.g., an enzymatic label or a radioisotope for detection of nucleic acid and/or expression of the gene in a cell. A wide variety of appropriate detectable markers are known in the art, including fluorescent, radioactive, enzymatic or other ligands, such as avidin/biotin, which are capable of giving a detectable signal. In preferred embodiments, one will likely desire to employ a fluorescent label or an enzyme tag, such as urease, alkaline phosphatase or peroxidase, instead of radioactive or other environmentally undesirable reagents. In the case of enzyme tags, calorimetric indicator substrates are known which can be employed to provide a means visible to the human eye or spectrophotometrically, to identify specific hybridization with complementary nucleic acid-containing samples. Briefly, this invention further provides a method for detecting a single-stranded polynucleotide identified by SEQ ID NO. 1 or its complement, by contacting target single-stranded polynucleotides with a labeled, single-stranded polynucleotide (a probe) which is a portion of the nucleotides shown in SEQ ID NO. 1 (or the corresponding complement) under conditions permitting hybridization (preferably moderately stringent hybridization conditions) of complementary single-stranded polynucleotides, or more preferably, under highly stringent hybridization conditions. Hybridized polynucleotide pairs are separated from un-hybridized, single-stranded polynucleotides. The hybridized polynucleotide pairs are detected using methods well known to those of skill in the art and set forth, for example, in Sambrook, et al. (1989) supra.

In an another aspect of this invention, the isolated polynucleotide encodes an oncogenic osteomalacia-related polypeptide, the polypeptide having the amino acid sequence of SEQ ID NO: 2 or an analog thereof having conservative amino acid substitutions. In a further aspect, the isolated polynucleotide of this invention encodes oncogenic osteomalacia-related mutein polypeptide, the mutein polypeptide having the amino acid sequence of SEQ ID NO: 2 or an analog thereof having non-conservative amino acid substitutions.

In one embodiment of the invention the oncogenic osteomalacia-related polynucleotide is isolated using the SAGE technique (Serial Analysis of Gene Expression or “SAGE,” disclosed in Velculescu, et al. (1995) Science 270:484-487 and U.S. Pat. No. 5,695,937). Using the SAGE tag for the polynucleotide of the present invention a full length cDNA encoding a oncogenic osteomalacia-related factor was isolated by hybridization to a cDNA library derived from an appropriate oncogenic osteomalacia cell. The additional embodiments of this invention can be isolated using the sequences provided in SEQ ID NOS: 1-2, and the methods described below or by homology searching publicly available databases.

Obtaining the Sequences to Practice the Invention

The polynucleotides and sequences used to practice the methods of this invention can be obtained using chemical synthesis, recombinant cloning methods, PCR, or any combination thereof. Methods of chemical polynucleotide synthesis are well known in the art and need not be described in detail herein. One of skill in the art can use the sequence data provided herein to obtain a desired polynucleotide by employing a DNA synthesizer or ordering from a commercial service.

Compositions containing the polynucleotides and sequences encoding the FRP-4 protein, in isolated form or contained within a vector or host cell may be delivered. When these compositions are to be used pharmaceutically, they are combined with a pharmaceutically acceptable carrier.

Suitable cell or tissue samples used for the methods of this invention encompass body fluid, solid tissue samples, tissue cultures or cells derived therefrom and the progeny thereof, and sections or smears prepared from any of these sources, or any other samples that may contain a neoplastic tumor tissue.

Polynucleotides of the invention can be isolated using the techniques described herein or replicated using PCR. The PCR technology is the subject matter of U.S. Pat. Nos. 4,683,195, 4,800,159, 4,754,065, and 4,683,202 and described in PCR: THE POLYMERASE CHAIN REACTION (Mullis et al. eds, Birkhauser Press, Boston (1994)) or MacPherson, et al. (1991) and (1994), supra, and references cited therein. Alternatively, one of skill in the art can use the sequences provided herein and a commercial DNA synthesizer to replicate the DNA. Still further, one of skill in the art can insert the polynucleotide into a suitable replication vector and insert the vector into a suitable host cell (prokaryotic or eukaryotic) for replication and amplification. The DNA so amplified can be isolated from the cell by methods well known to those of skill in the art. A process for obtaining polynucleotides by this method is further provided herein as well as the polynucleotides so obtained.

RNA can be obtained by first inserting a DNA polynucleotide into a suitable host cell. The DNA can be inserted by any appropriate method, e.g., by the use of an appropriate gene delivery vehicle (e.g., liposome, plasmid or vector) or by electroporation. When the cell replicates and the DNA is transcribed into RNA; the RNA can then be isolated using methods well known to those of skill in the art, for example, as set forth in Sambrook, et al. (1989) supra. For instance, mRNA can be isolated using various lytic enzymes or chemical solutions according to the procedures set forth in Sambrook, et al. (1989), supra or extracted by nucleic-acid-binding resins following the accompanying instructions provided by manufactures.

Polynucleotides exhibiting sequence complementarity or homology to SEQ ID NO. 1 find utility as hybridization probes. Since the full coding sequence of the transcript is known, any portion of this sequence or homologous sequences, can be used in the methods of this invention.

It is known in the art that a “perfectly matched” probe is not needed for a specific hybridization. Minor changes in probe sequence achieved by substitution, deletion or insertion of a small number of bases do not affect the hybridization specificity. In general, as much as 20% base-pair mismatch (when optimally aligned) can be tolerated. Preferably, a probe useful for detecting the aforementioned mRNA is at least about 80% identical to the homologous region More preferably, the probe is 85% identical to the corresponding gene sequence after alignment of the homologous region; even more preferably, it exhibits 90% identity.

These probes can be used in radioassays (e.g. Southern and Northern blot analysis) to detect, prognoses diagnose or monitor various neoplastic cells or tumor tissues containing these cells. The probes also can be attached to a solid support or an array such as a chip for use in high throughput screening assays for the detection of expression of the gene corresponding a polynucleotide of this invention. Accordingly, this invention also provides a probe comprising or corresponding to a polynucleotide of SEQ ID NO. 1, or its complement, or a fragment of SEQ ID NO. 1, attached to a solid support for use in high throughput screens.

The total size of fragment, as well as the size of the complementary stretches, will depend on the intended use or application of the particular nucleic acid segment. Smaller fragments will generally find use in hybridization embodiments, wherein the length of the complementary region may be varied, such as between at least 5 to 10 to about 100 nucleotides, or even full length according to the complementary sequences one wishes to detect.

Nucleotide probes having complementary sequences over stretches greater than 5 to 10 nucleotides in length are generally preferred, so as to increase stability and selectivity of the hybrid, and thereby improving the specificity of particular hybrid molecules obtained. More preferably, one can design polynucleotides having gene-complementary stretches of 10 or more or more than 50 nucleotides in length, or even longer where desired. Such fragments may be readily prepared by, for example, directly synthesizing the fragment by chemical means, by application of nucleic acid reproduction technology, such as the PCR technology with two priming oligonucleotides as described in U.S. Pat. No. 4,603,102 or by introducing selected sequences into recombinant vectors for recombinant production. A preferred probe is about 50-75 or more preferably, 50-100, nucleotides in length.

The polynucleotides described herein can serve as primers for the detection of genes or gene transcripts that are expressed in neoplastic cells associated with oncogenic osteomalacia. In this context, amplification means any method employing a primer-dependent polymerase capable of replicating a target sequence with reasonable fidelity. Amplification may be carried out by natural or recombinant DNA-polymerases such as T7 DNA polymerase, Klenow fragment of E. coli DNA polymerase, and reverse transcriptase. A preferred length of the primer is the same as that identified for probes, above.

A preferred amplification method is PCR. However, PCR conditions used for each reaction are empirically determined. A number of parameters influence the success of a reaction. Among them are annealing temperature and time, extension time, Mg²⁺ concentration, pH, and the relative concentration of primers, templates, and deoxyribonucleotides. After amplification, the resulting DNA fragments can be detected by agarose gel electrophoresis followed by visualization with ethidium bromide staining and ultraviolet illumination.

The methods of the invention can also employ the isolated polynucleotide encoding the FRP-4 protein operatively linked to a promoter of RNA transcription, as well as other regulatory sequences for replication and/or transient or stable expression of the DNA or RNA. As used herein, the term “operatively linked” means positioned in such a manner that the promoter will direct transcription of RNA off the DNA molecule. Examples of such promoters are SP6, T4 and T7. In certain embodiments, cell-specific promoters are used for cell-specific expression of the inserted polynucleotide. Vectors which contain a promoter or a promoter/enhancer, with termination codons and selectable marker sequences, as well as a cloning site into which an inserted piece of DNA can be operatively linked to that promoter are well known in the art and commercially available. For general methodology and cloning strategies, see GENE EXPRESSION TECHNOLOGY (Goeddel ed., Academic Press, Inc. (1991)) and references cited therein and VECTORS: ESSENTIAL DATA SERIES (Gacesa and Ramji, eds., John Wiley & Sons, N.Y. (1994)), which contains maps, functional properties, commercial suppliers and a reference to GenEMBL accession numbers for various suitable vectors. Preferable, these vectors are capable of transcribing RNA in vitro or in vivo.

Expression vectors containing these nucleic acids are useful to obtain host vector systems to produce proteins and polypeptides. It is implied that these expression vectors must be replicable in the host organisms either as episomes or as an integral part of the chromosomal DNA. Suitable expression vectors include plasmids, A preferred length of the primer is the same as that identified for probes, above viral vectors, including adenoviruses, adeno-associated viruses, retroviruses, cosmids, etc. Adenoviral vectors are particularly useful for introducing genes into tissues in vivo because of their high levels of expression and efficient transformation of cells both in vitro and in vivo. When a nucleic acid is inserted into a suitable host cell, e.g., a prokaryotic or a eukaryotic cell and the host cell replicates, the protein can be recombinantly produced. Suitable host cells will depend on the vector and can include mammalian cells, animal cells, human cells, simian cells, insect cells, yeast cells, and bacterial cells constructed using well known methods. See Sambrook, et al. (1989) supra. In addition to the use of viral vector for insertion of exogenous nucleic acid into cells, the nucleic acid can be inserted into the host cell by methods well known in the art such as transformation for bacterial cells; transfection using calcium phosphate precipitation for mammalian cells; or DEAE-dextran; electroporation; or microinjection. See Sambrook, et al. (1989) supra for this methodology. Thus, this invention also provides a host cell, e.g. a mammalian cell, an animal cell (rat or mouse), a human cell, or a procaryotic cell such as a bacterial cell, containing a polynucleotide encoding a protein or polypeptide or antibody.

When the vectors are used for gene therapy in vivo or ex vivo, a pharmaceutically acceptable vector is preferred, such as a replication-incompetent retroviral or adenoviral vector. Pharmaceutically acceptable vectors containing the nucleic acids of this invention can be further modified for transient or stable expression of the inserted polynucleotide. As used herein, the term “pharmaceutically acceptable vector” includes, but is not limited to, a vector or delivery vehicle having the ability to selectively target and introduce the nucleic acid into dividing cells. An example of such a vector is a “replication-incompetent” vector defined by its inability to produce viral proteins, precluding spread of the vector in the infected host cell. An example of a replication-incompetent retroviral vector is LNL6 (Miller, A. D. et al. (1989) BioTechniques 7:980-990). The methodology of using replication-incompetent retroviruses for retroviral-mediated gene transfer of gene markers is well established (Correll, et al. (1989) PNAS USA 86:8912; Bordignon (1989) PNAS USA 86:8912-52; Culver, K. (1991) PNAS USA 88:3155; and Rill, D. R. (1991) Blood 79(10):2694-700. Clinical investigations have shown that there are few or no adverse effects associated with the viral vectors, see Anderson (1992) Science 256:808-13.

Compositions containing the polynucleotides of this invention, in isolated form or contained within a vector or host cell are further provided herein. When these compositions are to be used pharmaceutically, they are combined with a pharmaceutically acceptable carrier.

Proteins

This invention provides uses for the FRP-4 protein or FRP-4 polypeptides expressed from the polynucleotides described above, which is intended to include wild-type and recombinantly produced polypeptides and proteins from prokaryotic and eukaryotic host cells, as well as muteins, analogs and fragments thereof. In some embodiments, the term also includes antibodies and anti-idiotypic antibodies. In one embodiment, these proteins or polypeptides are a phophatonin-related factor which modulates phosphatonin activity. Such polypeptides can be isolated or produced using the methods identified below.

It is understood that functional equivalents or variants of the wild-type polypeptide or protein also are within the scope of this invention, for example, those having conservative amino acid substitutions. Other analogs include fusion proteins comprising a protein or polypeptide.

The proteins and polypeptides described herein are obtainable by a number of processes well known to those of skill in the art, which include purification, chemical synthesis and recombinant methods. Full length proteins can be purified from a neoplastic cell or a tumor biopsy as identified above. Sources for purifying the protein can also be serum or urine samples from an individual, such as a patient with oncogenic osteomalacia. Proteins can be purified by methods such as immunoprecipitation with antibody, and standard techniques such as gel filtration, ion-exchange, reversed-phase, and affinity chromatography using a fusion protein as shown herein. For such methodology, see for example Deutscher et al. (1999) GUIDE To PROTEIN PURIFICATION: METHODS IN ENZYMOLOGY (Vol. 182, Academic Press). Accordingly, this invention also provides the processes for obtaining these proteins and polypeptides as well as the products obtainable and obtained by these processes.

The proteins and polypeptides also can be obtained by chemical synthesis using a commercially available automated peptide synthesizer such as those manufactured by Perkin Elmer/Applied Biosystems, Inc., Model 430A or 431A, Foster City, Calif., USA. The synthesized protein or polypeptide can be precipitated and further purified, for example by high performance liquid chromatography (HPLC). Accordingly, this invention also provides a process for chemically synthesizing the proteins of this invention by providing the sequence of the protein and reagents, such as amino acids and enzymes and linking together the amino acids in the proper orientation and linear sequence.

Alternatively, the proteins and polypeptides can be obtained by well-known recombinant methods as described, for example, in Sambrook, et al., (1989), supra, using the host cell and vector systems described above.

Also provided by this application are the polypeptides and proteins described herein conjugated to a detectable agent for use in the diagnostic methods. For example, detectably labeled proteins and polypeptides can be bound to a column and used for the detection and purification of antibodies. They also are useful as immunogens for the production of antibodies as described below. The proteins and fragments of this invention are useful in an in vitro assay system to screen for agents or drugs, which modulate cellular processes.

The FRP-4 proteins also can be combined with various liquid phase carriers, such as sterile or aqueous solutions, pharmaceutically acceptable carriers, suspensions and emulsions. Examples of non-aqueous solvents include propyl ethylene glycol, polyethylene glycol and vegetable oils. When used to prepare antibodies, the carriers also can include an adjuvant that is useful to non-specifically augment a specific immune response. A skilled artisan can easily determine whether an adjuvant is required and select one. However, for the purpose of illustration only, suitable adjuvants include, but are not limited to Freund's Complete and Incomplete, mineral salts and polynucleotides.

This invention also provides methods of using a pharmaceutical composition comprising any of a protein, analog, mutein, polypeptide fragment, antibody, antibody fragment or anti-idiotypic antibody of this invention, alone or in combination with each other or other agents, and an acceptable carrier. These compositions are useful for various diagnostic and therapeutic methods as described herein.

Antibodies

The present invention also envisions utilizing an antibody capable of specifically forming a complex with FRP-4 proteins or polypeptides as described above. The term “antibody” includes polyclonal antibodies and monoclonal antibodies. The antibodies include, but are not limited to mouse, rat, and rabbit or human antibodies.

Laboratory methods for producing polyclonal antibodies and monoclonal antibodies, as well as deducing their corresponding nucleic acid sequences, are known in the art, see Harlow and Lane (1988) supra and Sambrook, et al. (1989) supra. The monoclonal antibodies of this invention can be biologically produced by introducing protein or a fragment thereof into an animal, e.g., a mouse or a rabbit. The antibody producing cells in the animal are isolated and fused with myeloma cells or hetero-myeloma cells to produce hybrid cells or hybridomas. Accordingly, the hybridoma cells producing the monoclonal antibodies of this invention also are provided.

Thus, using the protein or fragment thereof, and well known methods, one of skill in the art can produce and screen the hybridoma cells and antibodies of this invention for antibodies having the ability to bind the proteins or polypeptides.

If a monoclonal antibody being tested binds with the protein or polypeptide, then the antibody being tested and the antibodies provided by the hybridomas of this invention are equivalent. It also is possible to determine without undue experimentation, whether an antibody has the same specificity as the monoclonal antibody of this invention by determining whether the antibody being tested prevents a monoclonal antibody of this invention from binding the protein or polypeptide with which the monoclonal antibody is normally reactive. If the antibody being tested competes with the monoclonal antibody of the invention as shown by a decrease in binding by the monoclonal antibody of this invention, then it is likely that the two antibodies bind to the same or a closely related epitope. Alternatively, one can pre-incubate the monoclonal antibody of this invention with a protein with which it is normally reactive, and determine if the monoclonal antibody being tested is inhibited in its ability to bind the antigen. If the monoclonal antibody being tested is inhibited then, in all likelihood, it has the same, or a closely related, epitopic specificity as the monoclonal antibody of this invention.

The term “antibody” also is intended to include antibodies of all isotypes. Particular isotypes of a monoclonal antibody can be prepared either directly by selecting from the initial fusion, or prepared secondarily, from a parental hybridoma secreting a monoclonal antibody of different isotype by using the sib selection technique to isolate class switch variants using the procedure described in Steplewski et al. (1985) Proc. Natl. Acad. Sci. 82:8653 or Spira et al. (1984) J. Immunol. Methods 74:307.

This invention also provides uses for biologically active fragments of the polyclonal and monoclonal antibodies described above. These “antibody fragments” retain some ability to selectively bind with its antigen or immunogen. Such antibody fragments can include, but are not limited to: Fab; Fab′; F(ab′)₂, Fv; and SCA.

A specific example of “a biologically active antibody fragment” is a CDR region of the antibody. Methods of making these fragments are known in the art, see for example, Harlow and Lane, (1988) supra.

The antibody compositions can be made even more compatible with a host system by minimizing potential adverse immune system responses. This may be accomplished in a variety of ways, including modifying the antibodies to create chimeric antibodies (e.g., antibodies in which the various domains of the antibodies' heavy and light chains are coded for by DNA from more than one species), such as humanized antibodies (Oi, et al. (1986) BioTechniques 4(3):214). This is accomplished by removing all or a portion of the Fc portion of a foreign species antibody or using an antibody of the same species as the host animal, for example, the use of antibodies from human/human hybridomas. Humanized antibodies (i.e., non immunogenic in a human) may be produced, for example, by replacing an immunogenic portion of an antibody with a corresponding, but non immunogenic portion (i.e., chimeric antibodies). Such chimeric antibodies may contain the reactive or antigen binding portion of an antibody from one species and the Fc portion of an antibody (non immunogenic) from a different species. Examples of chimeric antibodies, include but are not limited to, non-human mammal-human chimeras, rodent-human chimeras, murine-human and rat-human chimeras (Robinson et al., International Patent Application 184,187; Taniguchi M., European Patent Application 171,496; Morrison et al., European Patent Application 173, 494; Neuberger et al., PCT Application WO 86/01533; Cabilly et al., 1987 Proc. Natl. Acad. Sci. USA 84:3439; Nishimura et al., 1987 Canc. Res. 47:999; Wood et al., 1985 Nature 314:446; Shaw et al., 1988 J. Natl. Cancer Inst. 80: 15553, all incorporated herein by reference).

General reviews of “humanized” chimeric antibodies are provided by Morrison S., 1985 Science 229:1202 and by Oi et al., 1986 BioTechniques 4:214. Suitable “humanized” antibodies can be alternatively produced by CDR or CEA substitution (Jones et al., 1986 Nature 321:552; Verhoeyan et al., 1988 Science 239:1534; Biedleret al. 1988 J. Immunol. 141:4053, all incorporated herein by reference).

The antibodies or antigen binding fragments may also be produced by genetic engineering. The technology for expression of both heavy and light cain genes in E. coli is the subject the PCT patent applications; publication number WO 901443, WO901443, and WO 9014424 and in Huse et al., 1989 Science 246:1275-1281.

Alternatively, techniques described for the production of single chain antibodies (U.S. Pat. No. 4,946,778; Bird, 1988, Science 242, 423-426; Huston, et al., 1988, Proc. Natl. Acad. Sci. USA 85, 5879-5883; and Ward, et al., 1989, Nature 334, 544-546) can be adapted to produce single chain antibodies against Hcen-2 gene products. Single chain antibodies are formed by linking the heavy and light chain fragments of the Fv region via an amino acid bridge, resulting in a single chain polypeptide.

The isolation of other hybridomas secreting monoclonal antibodies with the specificity of the monoclonal antibodies of the invention can also be accomplished by one of ordinary skill in the art by producing anti-idiotypic antibodies (Herlyn, et al. (1986) Science 232:100). An anti-idiotypic antibody is an antibody which recognizes unique determinants present on the monoclonal antibody produced by the hybridoma of interest.

Idiotypic identity between monoclonal antibodies of two hybridomas demonstrates that the two monoclonal antibodies are the same with respect to their recognition of the same epitopic determinant. Thus, by using antibodies to the epitopic determinants on a monoclonal antibody it is possible to identify other hybridomas expressing monoclonal antibodies of the same epitopic specificity.

It is also possible to use the anti-idiotype technology to produce monoclonal antibodies which mimic an epitope. For example, an anti-idiotypic monoclonal antibody made to a first monoclonal antibody will have a binding domain in the hypervariable region which is the mirror image of the epitope bound by the first monoclonal antibody. Thus, in this instance, the anti-idiotypic monoclonal antibody could be used for immunization for production of these antibodies.

As used in this invention, the term “epitope” is meant to include any determinant having specific affinity for the monoclonal antibodies of the invention. Epitopic determinants usually consist of chemically active surface groupings of molecules such as amino acids or sugar side chains and usually have specific three dimensional structural characteristics, as well as specific charge characteristics.

The antibodies utilized in this invention can be linked to a detectable agent or label. There are many different labels and methods of labeling known to those of ordinary skill in the art.

The antibody-label complex is useful to detect the protein or fragments in a sample, using standard immunochemical techniques such as immunohistochemistry as described by Harlow and Lane (1988) supra. Competitive and non-competitive immunoassays in either a direct or indirect format are examples of such assays, e.g., enzyme linked immunoassay (ELISA) radioimmunoassay (RIA) and the sandwich (immunometric) assay. Those of skill in the art will know, or can readily discern, other immunoassay formats without undue experimentation.

The coupling of antibodies to low molecular weight haptens can increase the sensitivity of the assay. The haptens can then be specifically detected by means of a second reaction. For example, it is common to use haptens such as biotin, which reacts avidin, or dinitropherryl, pyridoxal, and fluorescein, which can react with specific anti-hapten antibodies. See Harlow and Lane (1988) supra.

Monoclonal antibodies also can be bound to many different carriers. Thus, this invention also envisions employing compositions containing the antibodies and another substance, active or inert. Examples of well-known carriers include glass, polystyrene, polypropylene, polyethylene, dextran, nylon, amylases, natural and modified celluloses, polyacrylamides, agaroses and magnetite. The nature of the carrier can be either soluble or insoluble for purposes of the invention. Those skilled in the art will know of other suitable carriers for binding monoclonal antibodies, or will be able to ascertain such, using routine experimentation.

Compositions containing the antibodies, fragments thereof or cell lines which produce the antibodies, are encompassed by this invention. When these compositions are to be used pharmaceutically, they are combined with a pharmaceutically acceptable carrier.

Cells

The invention provides methods for identification, characterization and modulation of a selected phenotype of a tumor mass isolated from a patient with oncogenic osteomalacia. Manipulation of selected cells is useful for practicing these methods of the invention.

Tumors from which sample cells can be obtained for use in the present invention are tumors originated from patients with symptoms of oncogenic osteomalacia. These include, but are not limited to, hemangiopericytomas and other tumors of mesenchymal origin, carcinoma of prostate and lung, fibrous dysplasia of bone, linear sebaceous naevus syndrome, neurofibromatosis, and oat cell carcinoma.

Tumor cells are typically obtained from a cancer patient by resection, biopsy, or endoscopic sampling; the cells may be used directly, stored frozen, or maintained or expanded in culture. Samples of both the tumor and the patient's blood or blood fraction should be thoroughly tested to ensure sterility before co-culturing of the cells. Standard sterility tests are known to those of skill in the art and are not described in detail herein. The tumor cells can be cultured in vitro to generate a cell line. Conditions for reliably establishing short-term cultures and obtaining at least 10⁸ cells from a variety of tumor types is described in Dillmar, et al. (1993) J. Immunother. 14:65-69. Alternatively, tumor cells can be dispersed from, for example, a biopsy sample, by standard mechanical means before use.

One aspect of the invention involves the comparison of transcript expression pattern between a sample cell and a control cell. The selection of the control cell is determined by the sample cell initially selected and the phenotype of interest. The control cell can be any of a counterpart normal cell type, a counterpart benign cell type, a counterpart non-neoplastic cell type and a non-neoplastic precursor of the neoplastic cell. For example, the sample cell can be a hemangiopericytoma cell isolated from a patient with oncogenic osteomalacia; the counterpart control cell can be a hemangiopericytoma cell isolated from a patient who does not have oncogenic osteomalacia.

Conditions for reliably establishing short-term cultures and obtaining at least 10⁸ cells from a variety of tumor types is described in Dillmar, et al. (1993) J. Immunother. 14:65-69. Alternatively, tumor cells can be dispersed from, for example, a biopsy sample, by standard mechanical means before use.

One aspect of the invention involves the comparison of transcript expression pattern between a sample cell and a control cell. The selection of the control cell is determined by the sample cell initially selected and the phenotype of interest. The control cell can be any of a counterpart normal cell type, a counterpart benign cell type, a counterpart non-neoplastic cell type and a non-neoplastic precursor of the neoplastic cell. For example, the sample cell can be a hemangiopericytoma cell isolated from a patient with oncogenic osteomalacia; the counterpart control cell can be a hemangiopericytoma cell isolated from a patient who does not have oncogenic osteomalacia.

Identification, Analysis, and Manipulation of Genetic Polymorphisms with SNP Technology

The isolated FRP-4 gene can be used to search for and identify single nucleotide polymorphisms (SNP's), which are mutant variants of the gene in the human population. Identification of such polymorphisms is useful to define human diseases to which mutations in the FRP-4 gene contribute and to perfect therapies for disease processes in which the protein encoded by the FRP-4 gene participates. Mutant variants of the gene identified in this manner can then be employed in the development, screening, and analysis of pharmaceutical agents to treat these diseases. Methods to detect such SNP's can be formatted to create diagnostic tests. Furthermore, various mutations in the gene which effect the response of different individuals to therapeutic agents can be identified and then diagnosed through analysis of SNP's, to guide the prescription of appropriate treatments. Also, SNP's identified in the FRP-4 gene can provide useful sequence markers for genetic tests to analyze other genes and mutations in the region of the genome where the FRP-4 gene is located. Thus it is useful to incorporate these SNP's into polymorphism databases.

Skilled practitioners of the art are familiar with an array of methods for identifying Bill and analyzing SNP's. High throughput DNA sequencing procedures such as sequencing by hybridization (Drmanac et al. (1993) Science 260:1649-52), minisequencing primer extension (Syvanen, (1999) Hum. Mutat. 13(1): 1-10), or other sequencing methods can be used to detect SNP's in defined regions of the gene. Alternatively, hybridization to oligonucleotides on DNA microarrays (Lipshutz et al. (1999) Nat. Genet. 21(1 Suppl.):20-4) analysis of single strand conformational polymorphisms in DNA or RNA molecules by various analytical methods (Nataraj (1999 Wiley & Sons, United Kingdom) pp:277-297; Dorin et al. (1992) Nature 359:211-215).) Electrophoresis 20(6):1177-85), PCR-based mutational analyses such as PCR with primers spanning the polymorphic sequence, or protection of SNP-containing oligonucleotides from nuclease protection such as by use of the bacterial mutS protein can be employed. Many sophisticated high-throughput technologies based on methods such as automated capillary electrophoresis (Larsen et al. (1999) Hum. Mutat. 13(4):318-27), time-of-flight mass spectroscopy (Li et al. (1999) supra, high density micro-arrays (Sapolsky et al. (1999) Genet. Anal. 14(5-6):187-92), semiconductor microchips (Gilles et al. (1999) Nat. Biotechnol. 17(4):365-70), and others have been demonstrated that can be employed with the FRP-4 gene to perform the uses described above.

All books, articles, and patents referenced herein are incorporated by reference. The following examples, are intended to illustrate, but not limit this invention.

### EXAMPLES

1. Functional Analysis of the FRP-4 Gene

A full length cDNAs encoding the FRP-4 protein was inserted into an expression vector and the FRP-4 protein was expressed in mammalian culture cells using standard cloning strategies. Stable cell lines that can be rapidly scaled for production were also established. In vitro expressed FRP-4 was then produced as a secreted molecule in conditioned culture medium and prepared for functional assays measuring phosphate reabsorption.

Phosphate Transport Assay

The phosphate transport modulating activity of the FRP-4 protein was analyzed using methods and techniques known in the art. Specifically, sodium-dependent phosphate uptake was measured in opossum kidney (OK) cells according to the methods described in Cai et al. (1994) New Engl. J. Med. 330:1645-1649. Briefly, OK cells were cultured until becoming confluent, harvested and then re-seeded at a density of 1×10⁵ cells per 24 well dish. The cells were then re-grown for several days past the time they become confluent and then re-fed with medium containing the FRP-4 protein as well as medium containing a variety of alternative experimental and control factors. After the incubation period extending from 3 to 48 hours, the medium was removed and the plated cells were re-fed with transport medium containing ³²P-labeled dibasic potassium phosphate and incubated at 37° C. for 5 minutes. The cells were then washed, harvested and radioactivity measured via a scintillation counter to monitor uptake of ³²P.

Results of the OK phosphate transport assay performed on conditioned medium containing the FRP-4 protein and control samples showed that conditioned medium that contained the FRP-4 protein induced a statistically significant reduction in phosphate uptake by the OK cells in comparison with control samples that did not contain this factor.

It is to be understood that while the invention has been described in conjunction with the above embodiments, that the foregoing description and the following examples are intended to illustrate and not limit the scope of the invention. Other aspects, advantages and modifications within the scope of the invention will be apparent to those skilled in the art to which the invention pertains.

What is claimed is:

1. A method of modulating phosphate homeostasis in a subject comprising altering the activity of a polypeptide encoded by the FRP-4 gene within the subject.

2. The method of claim 1, wherein phosphate homeostasis is modulate by delivering to the subject an effective amount of an agent that alters the activity of a polypeptide encoded by the FRP-4 gene.

3. A method of modulating phosphate homeostasis in a subject comprising altering the expression of a polynucleotide encoding FRP-4 polypeptide within the subject.

4. A method for modulating renal phosphate transport in a subject, comprising delivering to the subject an effective amount of an agent that alters the activity of a polypeptide encoded by the FRP-4 gene.

5. A method of reducing phosphate re-absorption in a subject comprising delivering to the subject an effective amount of the FRP-4 protein.

6. A method of reducing phosphate re-absorption in a subject comprising delivering to the subject an effective amount of a polynucleotide encoding the FRP-4 protein.

7. A method of screening for candidate therapeutic agents that modulate the expression of the FRP-4 gene comprising contacting a target cell with a test agent and monitoring expression of the FRP-4 gene, wherein a test agent which modifies the expression of the FRP-4 gene is a candidate therapeutic agent.

8. The method of claim 7, wherein the candidate agent is a biological or chemical compound selected from the group consisting a polypeptide, a polynucleotide, a ribozyme, and a small organic molecule.

9. A method of screening for candidate agents capable of altering the biological activity of a polypeptide encoded by the FRP-4 gene, comprising contacting a target cell expressing a FRP-4 polypeptide with a test agent and monitoring activity of the expressed polypeptide product, wherein a test agent which modifies the activity of the polypeptide is a candidate agent.

10. The method of claim 9, wherein the candidate agent is a biological or chemical compound selected from the group consisting of a polypeptide, a polynucleotide, a ribozyme, or a small organic molecule.

11. A method of screening for candidate agents that modulate the activity of the FRP-4 protein comprising contacting a target cell with a candidate agent and monitoring the activity of the FRP-4 protein, wherein a candidate agent which modifies the activity of the FRP-4 protein is a candidate therapeutic agent.

12. The method of claim 11, wherein the candidate agent is a biological or chemical compound selected from the group consisting of a polypeptide, a polynucleotide, a ribozyme, or a small organic molecule.

13. A method of screening for candidate ligand that modulate the activity of the FRP-4 protein comprising contacting a target cell with a candidate agent and monitoring the activity of the FRP-4 protein, wherein a candidate agent which modifies the activity of the FRP-4 protein is a candidate ligand.

14. The method of claim 13, wherein the candidate agent is a biological or chemical compound selected from the group consisting of a polypeptide, a polynucleotide, a ribozyme, or a small organic molecule.
